(19) United States
(12) Patent Application Publication
(10) Pub. No.: US 2010/0113791 A1
(43) Pub. Date: May 6, 2010

(54) PREPARATION OF PRECURSORS OF CARBENES OF CAAC TYPE AND PREPARING SAID CARBENES THEREFROM

(76) Inventor: Gérard Mignani, Lyon (FR)

Correspondence Address: BUCHANAN, INGERSOLL & ROONEY PC, POST OFFICE BOX 1404, ALEXANDRIA, VA, 22313-1404, US

(21) Appl. No.: 12/595,161

(22) PCT Filed: Apr. 9, 2008
(86) PCT No.: PCT/EP2008/054295

§ 371 Date: Dec. 23, 2009

(30) Foreign Application Priority Data
Apr. 12, 2007 (FR) 07/02665

(51) Int. Cl. C07D 209/54; C07D 207/20; C07D 211/14; C07C 251/04

(52) U.S. Cl. 546/192; 548/408; 548/565; 548/411; 564/248

(57) ABSTRACT

Precursors of carbenes of CAAC type (Cyclic)(Alkyl)(Amino)(Carbenes) are prepared and carbenes are produced therefrom; novel synthesis intermediates are provided for preparing the precursors of CAAC-type carbenes.

A subject matter of the present invention is a process for the preparation of precursors of carbenes of CAAC (Cyclic)(Alkyl)(Amino)(Carbenes) type and their use in preparing said carbenes.

The invention is targeted at the use of novel synthetic intermediates in preparing the precursors of carbenes of CAAC type.

Carbenes are compounds capable of being used as organocatalysts or as ligands for metals such as palladium, platinum, nickel, ruthenium, rhodium, iridium, copper and iron, thus forming stable organo-metallic complexes which can be used as catalysts for organic reactions, in particular in coupling reactions between an electrophilic reactant, generally an aromatic compound carrying a leaving group (such as halogen, sulfonic ester, azonium, and the like), and a nucleophilic compound contributing a carbon atom or a heteroatom capable of replacing the leaving group, thus creating a C—C or C-HE bond (HE being a heteroatom, for example N, O, S, Si, and the like).

One of the categories of carbenes is composed of carbenes comprising a cyclic alkylamino unit, known as “CAACs”, which abbreviation originates from the name Cyclic (Alkyl) (Amino) Carbenes.

CAACs are known compounds described in the literature, in particular in WO 2006/138166.

An example of this type of carbene is given by the following formula:

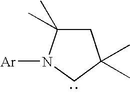

A route for the synthesis of said carbene is represented by the following reaction scheme:

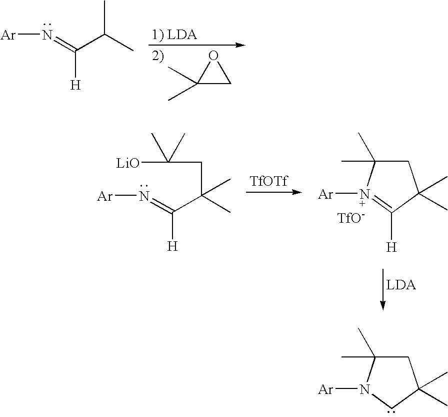

The drawback of such a process is that it is difficult to transfer to an industrial scale as it involves LDA, the lithium salt of diisopropylamine which is a strong base not used industrially. This route also involves an epoxide, which is a toxic and expensive reactant. Furthermore, this preparation process is not universal and does not apply, for example, when the aryl group is replaced by a menthyl group.

The applicant company specifically provides a process which makes it possible to avoid this disadvantage by involving a different intermediate.

A subject matter of the present invention is the use, as intermediate in the manufacture of a precursor of CAAC carbene, of a compound corresponding to the following formula:

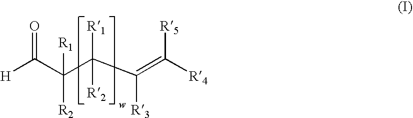

in said formula:
w is equal to 1 or 2,
R₁ and R₂, which are identical or different, represent an alkyl, alkenyl, alkynyl, cycloalkyl, aryl, aralkyl, alkoxy, alkenyloxy, alkynyloxy, aryloxy or alkoxycarbonyl group,
or R₁ and R₂ can be bonded together to form a spiro ring comprising from 3 to 18 atoms,
R′₁, R′₂, R′₃, R′₄ and R′₅, which are identical or different, represent a hydrogen atom or an alkyl, cycloalkyl, aryl or aralkyl group.

In the context of the invention, “alkyl” is understood to mean a linear or branched C₁-C₁₅, preferably C₁-C₁₀ and more preferably still C₁-C₄ hydrocarbon chain. Examples of preferred alkyl groups are in particular methyl, ethyl, propyl, isopropyl, butyl, isobutyl or tert-butyl.

“Alkoxy” is understood to mean an alkyl-O— group in which the alkyl term has the meaning given above. Preferred examples of alkoxy groups are the methoxy and ethoxy groups.

“Alkoxycarbonyl” refers to the alkoxy-C(O)— group in which the alkoxy group has the definition given above.

“Alkenyl” is understood to mean a linear or branched C₂-C₈, preferably C₂-C₆ and more preferably still C₂-C₄ hydrocarbon chain comprising a double bond. Examples of preferred alkenyl groups are in particular the vinyl, 1-propenyl, 2-propenyl, isopropenyl, 1-butenyl, 2 butenyl, 3-butenyl and isobutenyl groups.

“Alkynyl” is understood to mean a linear or branched C₂-C₈, preferably C₂-C₆ and more preferably still C₂-C₄ hydrocarbon chain comprising a triple bond. Examples of preferred alkynyl groups are in particular ethynyl, 1-propynyl, 1-butynyl and 2-butynyl groups.

“Alkenyloxy” and “alkynyloxy” are respectively understood to, mean an alkenyl-O— and alkynyl-O— group in which the alkenyl and alkynyl terms have the meanings given above.

“Cycloalkyl” is understood to mean a cyclic hydrocarbon group which is a C₃-C₁₀ monocyclic hydrocarbon group, preferably a cyclopentyl or cyclohexyl group or a C₄-C₁₈ polycyclic (bi- or tricyclic) hydrocarbon group, in particular an adamantyl or norbornyl group.

“Spiro ring of cycloalkane type” is understood to mean, as defined above, a C₃-C₁₈, preferably C₃-C₁₀, mono- or polycyclic structure.

“Aryl” is understood to mean a C₆-C₂₀ aromatic mono- or polycyclic group, preferably mono- or bicyclic group, preferably phenyl or naphthyl. When the group is polycyclic, that is to say when it comprises more than one cyclic unit, the cyclic units are able to be fused in pairs or attached in pairs via a bonds. Examples of (C₆-C₁₈)aryl groups are in particular phenyl and naphthyl.

“Aryloxy” is understood to mean an aryl-O— group in which the aryl group has the meaning given above.

“Arylalkyl” is understood to mean a linear or branched hydrocarbon group carrying an aromatic C₇-C₁₂ monocyclic ring, preferably a benzyl group: the aliphatic chain comprising 1 or 2 carbon atoms.

It should be noted that, provided that one of the R₁, R₂, R′₁, R′₂, R′₃, R′₄ and R′₅ groups comprises a ring, the latter can be substituted by one or more substituents, preferably two or three substituents. The substituent can be of any nature provided that it does not interfere with the synthesis of the CAAC. Mention may be made in particular, as preferred examples of substituent symbolized by Rₜ, of alkyl, alkoxy, alkenyl, alkynyl, cycloalkyl, aryl, aralkyl or amino groups, it being possible for the amino to be substituted by alkyl or cycloalkyl groups; a nitrile group; a halogen atom, preferably a chlorine or fluorine atom; or a haloalkyl group, preferably a perfluoromethyl group.

The compounds of formula (I) are denoted “intermediate compounds” as they are intermediates in the manufacture of precursors of carbenes.

The preferred intermediate compounds correspond to the formula (Ia) or (Ib):

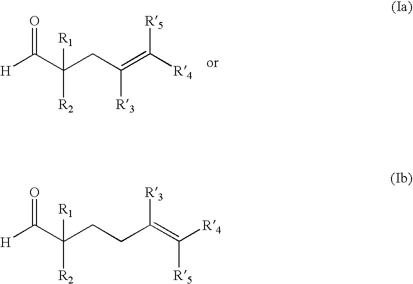

in which:
R₁ and R₂, which are identical or different, represent an alkyl group, or an optionally substituted aryl group, preferably an optionally substituted phenyl or naphthyl group,
or else R₁ and R₂ are bonded together to form a spiro ring of mono- or polycyclic cycloalkane type,
R′₃, R′₄ and R′₅, which are identical or different, represent an alkyl group,
R′₄ and R′₅ also represent a hydrogen atom.

In said formulae, when R₁ and R₂ represent an optionally substituted aryl group, the substituent or substituents can be as defined above (Rₜ) but are preferably alkyl groups and/or alkoxy groups and/or aryl groups, preferably phenyl groups.

The intermediate compounds prepared correspond more preferably to the formula (Ia) or (Ib) in which:
R₁ and R₂, which are identical or different, represent a linear or branched alkyl group having from 1 to 4 carbon atoms or a phenyl group or a substituted phenyl group,
or R₁ and R₂ are bonded together to form a cyclopentane, a cyclohexane or a norbornane,
R′₃, R′₄ and R′₅, which are identical or different, represent a linear or branched alkyl group having from 1 to 4 carbon atoms and R′₄ and R′₅ also represent a hydrogen atom.

Another subject matter of the invention is the process for the preparation of a precursor of carbene CAAC, also denoted “iminium salt” of formula (VI):

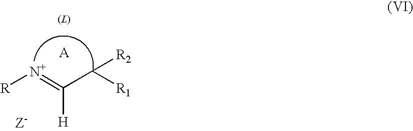

in said formula:
R represents an alkyl, cycloalkyl, aryl, aralkyl or heteroaryl group,
R₁ and R₂, which are identical or different, represent an alkyl, alkenyl, alkynyl, cycloalkyl, aryl, aralkyl, alkoxy, alkenyloxy, alkynyloxy, aryloxy or alkoxycarbonyl group,
or R₁ and R₂ can be bonded together to form a spiro ring comprising from 3 to 18 atoms,
A represents a ring comprising 5 or 6 atoms, at least one of the atoms of which is a nitrogen atom as represented,
L is a divalent group corresponding to the following formula:

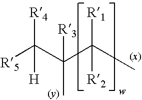

w is a number equal to 1 or 2,

R′₁, R′₂, R′₂, R′₄ and R′₅, which are identical or different, represent a hydrogen atom or an alkyl, cycloalkyl, aryl or aralkyl group,
(x) and (y) respectively pinpoint the two bonds established between the carbon atom carrying the R₁ and R₂ groups and the nitrogen atom carrying the R group,
Z is an anion,
   characterized in that it comprises at least one stage of reaction:
of the compound of formula (I):

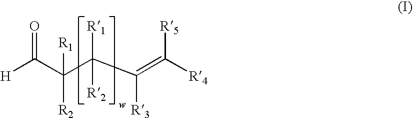

in said formula:
R₁, R₂, R′₁, R′₂, R′₂, R′₄, R′₅ and w have the meanings given above,
and of a primary amine of formula (V):

R—NH₂ (V)

in said formula:
R represents an alkyl, cycloalkyl, aryl, aralkyl or heteroaryl group,
   resulting in the formation of an imine corresponding to the formula (IV):

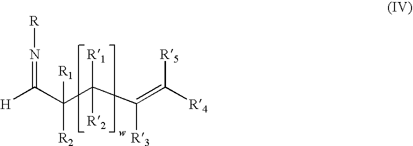

in said formula:
R, R₁, R₂, R′₁, R′₂, R′₃, R′₄, R′₅ and w have the meanings given above.

According to a preferred implementation of the process of the invention, the intermediate compound of formula (I) is prepared by reaction of an aldehyde having at least one hydrogen atom in the a position with respect to the carbonyl group and an unsaturated reactant, carrying a leaving group, in a two-phase medium, in the presence of a strong base and of a phase transfer catalyst.

Another subject matter of the invention is the process for the preparation of a precursor of carbene CAAC of formula (VI) according to a stage of cyclization of the compound of formula (IV).

To facilitate understanding of the account of the invention, the reaction scheme of the process of the invention is given below, without, however, limiting the scope of the invention to this scheme.

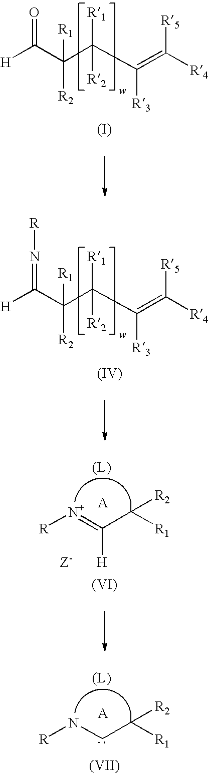

In said formulae, the various symbols have the meanings already explained or explained subsequently.

In accordance with the process of the invention, the preparation of the intermediate compound of formula (I) is carried out according to a preparation process which comprises the reaction of an aldehyde having at least one hydrogen atom in the a position with respect to the carbonyl group and an unsaturated reactant, carrying a leaving group, in a two-phase medium, in the presence of a strong base and of a phase transfer catalyst.

More specifically, the intermediate compound of formula (I) is obtained according to the reaction:
of an aldehyde of formula (II):

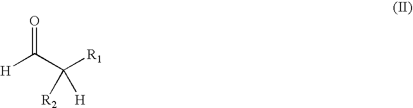

in said formula:
R₁ and R₂, which are identical or different, represent an alkyl, alkenyl, alkynyl, cycloalkyl, aryl, aralkyl, alkoxy, alkenyloxy, alkynyloxy, aryloxy or alkoxycarbonyl group,
or R₁ and R₂ can be bonded together to form a spiro ring comprising from 3 to 18 atoms,
   and
an unsaturated reactant carrying a leaving group:

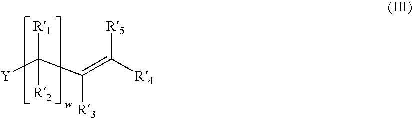

in said formula:
w is a number equal to 1 or 2,
R′₁, R′₂, R′₃, R′₄ and R′₅, which are identical or different, represent a hydrogen atom or an alkyl, cycloalkyl, aryl or aralkyl group,
Y represents a leaving group.

In the formula (III), Y represents a leaving group chosen from bromine, chlorine or a sulfonic ester group of formula —OSO₂—Rₑ, in which Rₑ is a hydrocarbon group.

In the formula of the sulfonic ester group, Rₑ is a hydrocarbon group of any nature. However, given that Y is a leaving group, it is advantageous from an economic viewpoint for Rₑ to be simple in nature and more particularly to represent a linear or branched alkyl group having from 1 to 4 carbon atoms, preferably a methyl or ethyl group, but it can also represent, for example, a phenyl or tolyl group or a trifluoromethyl group. Among the Y groups, the preferred group is a triflate group, which corresponds to an Rₑ group representing a trifluoromethyl group.

The choice is preferably made, as preferred leaving groups, of a chlorine atom.

The amount of unsaturated reactant of formula (III) to be employed, expressed with respect to the amount of the aldehyde of formula (II), is at least equal to stoichiometry. Thus, the ratio of the number of moles of unsaturated reactant of formula (III) to the number of moles of aldehyde of formula (II) varies between 1 and 1.5 and is preferably between 1.2 and 1.3.

The process of the invention thus involves a base, which can be inorganic or organic.

The base used has to be sufficiently strong to make possible the anionization of the carbon-hydrogen bond of the aldehyde.

“Strong base” is understood to mean a base, the pKa of the conjugate acid of which is greater than or equal to 9, preferably between 10 and 14.

The pKa is defined as the ionic dissociation constant of the acid/base pair when water is used as solvent.

For the choice of a base having a pKa as defined by the invention, reference may be made, inter alia, to the Handbook of Chemistry and Physics, 66th edition, pp. D-161 and D-162.

To do this, recourse is had to an inorganic base, such as an alkali metal or alkaline earth metal hydroxide, preferably sodium hydroxide, potassium hydroxide or lithium hydroxide, or an alkali metal phosphate or hydrogen phosphate, preferably sodium phosphate or potassium phosphate, or to an organic base, preferably sodium methoxide.

It is also possible to resort to a quaternary ammonium hydroxide.

Use is preferably made, as examples of quaternary ammonium hydroxides, of tetraalkylammonium or trialkylbenzylammonium hydroxides, the alkyl groups of which, which are identical or different, represent a linear or branched alkyl chain having from 1 to 12 carbon atoms, preferably from 1 to 6 carbon atoms.

The choice is preferably made of tetramethylammonium hydroxide, tetraethylammonium hydroxide, tetrabutyl-ammonium hydroxide or trimethylbenzylammonium hydroxide.

For economic reasons, the choice is preferably made, from all the bases, of sodium hydroxide or potassium hydroxide.

The base is employed without distinction in the form of a powder or in the form of an aqueous solution.

The concentration of the starting basic solution is not critical. The alkali metal hydroxide solution employed has a concentration generally of between 10 and 50% by weight.

Generally, the amount of base employed, expressed by the ratio of the number of moles of OH⁻ ions to the number of moles of compound of formula (II), varies between 2 and 5, preferably between 3 and 4.

The reaction of the compounds of formulae (II) and (III) is carried out in the absence or in the presence of an organic solvent.

The organic solvent is chosen so that it is inert under the reaction conditions of the invention.

Mention may be made in particular, as examples of organic solvent suitable for the process of the invention, of aliphatic, cycloaliphatic or aromatic hydrocarbons and more particularly hexane, cyclohexane, methylcyclohexane, petroleum fractions of petroleum ether type, aromatic hydrocarbons, such as, in particular, toluene, xylene, cumene, mesitylene or petroleum fractions composed of a mixture of alkylbenzenes, in particular fractions of the Solvesso type, or decalin.

Use may also be made of a mixture of organic solvents.

The amount of solvent employed is determined so that the concentration of the compound of formula (II) in the organic solvent is preferably between 2 and 10 mol/liter, preferably approximately 3 mol/liter.

The reaction between the compounds of formulae (II) and (III) is carried out in the presence of a phase transfer catalyst.

The expression “phase transfer catalyst” is understood to mean a catalyst capable of causing the anion to pass from the aqueous phase to the organic phase.

In the process of the invention, recourse may be had to known phase transfer catalysts, in particular those described in the work by Jerry March, Advanced Organic Chemistry, 4th edition, John Wiley & Sons, 1992, p. 362 et seq.

The onium salts capable of being used in the process according to the invention are those having onium ions deriving in particular from nitrogen, phosphorus, sulfur, oxygen, carbon or iodine coordinated to hydrocarbon residues. The onium ions deriving from nitrogen or phosphorus will be tetracoordinated, the onium ions deriving from sulfur, oxygen, carbon or S═O will be tricoordinated, while the onium ions deriving from iodine will be dicoordinated.

The hydrocarbon residues coordinated to these various elements are alkyl, alkenyl, aryl, cycloalkyl or aralkyl groups which are optionally substituted, it being possible for two coordinated hydrocarbon residues to together form a single divalent group.

The nature of the anions bonded to these organic cations is not of critical importance. All “hard” or “borderline” bases are suitable as anion. “Hard” or “borderline” base is understood to mean any anion corresponding to the conventional definition given by R. Pearson in Journal of Chem. Ed., 45, pages 581-587 (1968), the terms “hard” and “borderline” respectively having the meanings of these terms used in this reference.

Particularly suitable among the onium ions which can be used in the present process of the invention are those corresponding to the following general formula:

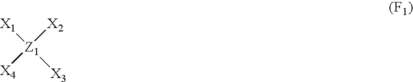

in said formula:
Z₁ represents N or P,
X₁, X₂, X₃ and X₄, which are identical or different, represent:
   a linear or branched alkyl group having from 1 to 16 carbon atoms which is optionally substituted by one or more phenyl, hydroxyl, halogen, nitro, alkoxy or alkoxycarbonyl groups or atoms, the alkoxy groups having from 1 to 4 carbon atoms,
   a linear or branched alkenyl group having from 2 to 12 carbon atoms,
   an aryl group having from 6 to 10 carbon atoms which is optionally substituted by one or more alkyl groups having from 1 to 4 carbon atoms, alkoxy groups, alkoxycarbonyl groups, the alkoxy group having from 1 to 4 carbon atoms, or halogen atoms,
   it being possible for two of said groups X₁ to X₄ together to form a linear or branched alkylene, alkenylene or alkadienylene group having from 3 to 6 carbon atoms.

Mention may be made, among the “hard” or “borderline” anions which can form the anion of said onium salts, of the ions: F⁻, ClO₄⁻, PF₆⁻, BF₄⁻, SnCl₆⁻, SbCl₆⁻, B(Ph)₄⁻, PO₄³⁻, HPO₄²⁻, H₂PO₄⁻, CH₃SO₃⁻, Ph-SO₃⁻, HSO₄⁻, NO₃⁻, SO₄²⁻, Cl⁻, Br⁻, I⁻ and OH⁻, Ph representing a phenyl group, and all the other anions corresponding to the “hard” or “borderline” basic definition of Pearson.

For reasons of operating convenience, said anions can be chosen from PO₄³⁻, HPO₄²⁻, H₂PO₄⁻, CH₃SO₃⁻, Ph-SO_{3 NO}₃⁻, SO₄²⁻, PF₆⁻, Br⁻ and I⁻, Ph having the above meaning.

Recourse will advantageously be had to the anions Br⁻, Cl⁻, OH⁻ and HSO₄⁻.

Mention may be made, as examples of onium ions corresponding to the formula (F₁), of the cations:
tetramethylammonium,
triethylmethylammonium,
tributylmethylammonium,
trimethylpropylammonium,
tetraethylammonium,
tetrabutylammonium,
dodecyltrimethylammonium,
methyltrioctylammonium,
heptyltributylammonium,
tetrapropylammonium,
tetrapentylammonium,
tetrahexylammonium,
tetraheptylammonium,
tetraoctylammonium,
tetradecylammonium,
butyltripropylammonium,
methyltributylammonium,
pentyltributylammonium,
methyldiethylpropylammonium,
ethyldimethylpropylammonium,
tetradodecylammonium,
tetraoctadecylammonium,
hexadecyltrimethylammonium,
benzyltrimethylammonium,
benzyldimethylpropylammonium,
benzyldimethyloctylammonium,
benzyltributylammonium,
benzyltriethylammonium,
phenyltrimethylammonium,
benzyldimethyltetradecylammonium,
benzyldimethylhexadecylammonium,
dimethyldiphenylammonium,
methyltriphenylammonium,
(buten-2-yl)triethylammonium,
N,N-dimethyltetramethyleneammonium,
N,N-diethyltetramethyleneammonium,
tetramethylphosphonium,
tetrabutylphosphonium,
ethyltrimethylphosphonium,
trimethylpentylphosphonium,
octyltrimethylphosphonium,
dodecyltrimethylphosphonium,
trimethylphenylphosphonium,
diethyldimethylphosphonium,
dicyclohexyldimethylphosphonium,
dimethyldiphenylphosphonium,
cyclohexyltrimethylphosphonium,
triethylmethylphosphonium,
methyltri(isopropyl)phosphonium,
methyltri(n-propyl)phosphonium,
methyltri(n-butyl)phosphonium,
methyltri(2-methylpropyl)phosphonium,
methyltricyclohexylphosphonium,
methyltriphenylphosphonium,
methyltribenzylphosphonium,
methyltri(4-methylphenyl)phosphonium,
methyltrixylylphosphonium,
diethylmethylphenylphosphonium,
dibenzylmethylphenylphosphonium,
ethyltriphenylphosphonium,
tetraethylphosphonium,
ethyltri(n-propyl)phosphonium,
triethylpentylphosphonium,
hexadecyltributylphosphonium,
ethyltriphenylphosphonium,
n-butyltri(n-propyl)phosphonium,
butyltriphenylphosphonium,
benzyltriphenylphosphonium,
(β-phenylethyl)dimethylphenylphosphonium,
tetraphenylphosphonium,
triphenyl(4-methylphenyl)phosphonium,
tetrakis(hydroxymethyl)phosphonium.

Preference will generally be given, among the onium ions which can be used in the context of the present process, to quaternary ammonium ions or quaternary phosphonium ions.

Ammonium or phosphonium ions in which the four groups are alkyl groups having from 1 to 5 carbon atoms or a benzyl group are very particularly well suited.

As regards the choice of the anion, preference is given to Br⁻, Cl⁻, OH⁻, or HSO₄⁻.

The catalysts entirely well suited to the present invention are tributylbenzylammonium or -phosphonium chloride or bromide, tetramethylammonium or -phosphonium chloride or bromide, tetraethylammonium or -phosphonium chloride or bromide, or tetrabutylammonium or -phosphonium chloride or bromide.

Benzyltributylammonium chloride or bromide is particularly preferred, the chlorinated derivative being more particularly preferred.

The onium salt can be introduced during the process of the invention, in the solid form or in the form of a solution in one of its solvents, generally water.

Mention may be made, as other examples of phase transfer catalysts capable of being employed in the process of the invention, of tri(ether amine)s, in particular described in FR-A 2 455 570, which correspond to the following formula:

N+A-O(—B—O—)—ₙ—Rₐ]₃(F₂)

in said formula:
Rₐ represents an alkyl group having from 1 to 24 carbon atoms, a cyclohexyl group, a phenyl group or an alkylphenyl group comprising from 7 to 12 carbon atoms,
A and B, which are alike or different, represent a linear alkanediyl group comprising 2 or 3 carbon atoms which are optionally substituted by a methyl or ethyl group,
n is an integer between 0 and 4.

Mention may be made, as preferred examples of catalysts corresponding to the formula (F₂), of tris(3,6-dioxaheptyl)amine sold under the name TDA-1.

As regards the amount of catalyst used, this varies advantageously so that the molar ratio of said catalyst to the compound of formula (II) varies between 0.01 and 0.10, preferably between 0.01 and 0.05. The upper limit is not critical and can be greatly exceeded without disadvantage as the catalyst can be optionally recycled at the end of the reaction.

A preferred embodiment comprises the introduction in parallel of the compounds of formulae (II) and (III) into a medium comprising the solvent (water and/or organic solvent), the base and the phase transfer catalyst.

The compounds of formulae (II) and (III) are advantageously introduced gradually, fractionwise or continuously. The duration of addition is, for example, between 2 and 10 hours, preferably between 4 and 5 hours.

The coupling reaction of the compounds of formulae (II) and (III) is generally carried out at a temperature of between 50° C. and 100° C., preferably between 70° C. and 80° C.

The process of the invention is carried out at atmospheric pressure but preferably under a controlled atmosphere of inert gases, such as nitrogen, or rare gases, for example argon. A pressure slightly greater than or less than atmospheric pressure may be suitable.

At the end of the reaction, the intermediate compound corresponding to the formula (I):

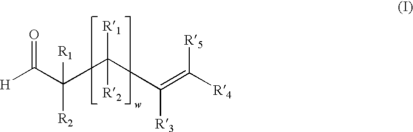

in said formula:
R₁, R₂, R′₁, R′₂, R′₃, R′₄, R′₅ and w have the meanings given above, is obtained.

Said compound is recovered from the reaction medium, which comprises an organic phase comprising the addition product of formula (I) and the reactants (II) or (III) in excess and an aqueous phase comprising the salts resulting from the reaction.

The organic and aqueous phases are separated.

The aqueous phase can optionally be washed once or twice using an organic solvent, preferably the solvent chosen for the reaction.

The organic phases are combined and then a normal drying operation is preferably carried out over a dehydrating agent, for example sodium sulfate or magnesium sulfate.

After separating from the dehydrating agent, preferably by filtration, the reaction medium is generally concentrated by evaporation and then the intermediate compound of formula (I) is recovered in conventional fashion, preferably by distillation.

In accordance with the process of the invention, the compound of formula (I) is used as intermediate in the manufacture of a compound of imine type of formula (IV):

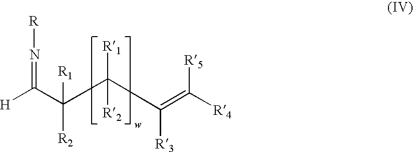

in said formula (IV),
R₁, R₂, R′₁, R′₂, R′₃, R′₄, R′₅ and w have the meanings given above,
R represents an alkyl, cycloalkyl, aryl, aralkyl or heteroaryl group, characterized in that it is obtained by reaction, in the presence of a strong acid, of the compound of formula (I) with a primary amine of formula (V):

R—NH₂ (V)

in said formula:
R represents an alkyl, cycloalkyl, aryl, aralkyl or heteroaryl group.

“Heteroaryl” is understood to mean an aryl group as defined above, 1 to 3 carbon atoms of which can be replaced by a heteroatom, preferably oxygen or nitrogen.

In this stage for the preparation of the compound of imine type of formula (IV), a primary amine which corresponds to the formula (V) is involved.

The R group is a hydrocarbon group which is preferably sterically hindered.

It is thus more particularly a linear or branched alkyl group having from 1 to 12 carbon atoms, and more preferably a branched alkyl group having from 3 to 12 carbon atoms, preferably an isopropyl or tert-butyl group; a cycloalkyl group having from 5 to 7 carbon atoms; an aryl group having from 6 to 20 carbon atoms and more preferably a phenyl group or a phenyl group carrying from 1 to 3 substituents, such as, for example, C₁-C₄ alkyl or alkoxy; or a naphthyl group or a naphthyl group carrying 1 or 2 substituents, such as, for example, C₁-C₄ alkyl or alkoxy.

The substituents on the phenyl group are preferably in the ortho and/or para and/or ortho′ position with respect to the amino group.

The substituents on the naphthyl group are preferably in the ortho and/or ortho′ position with respect to the amino group.

Mention may in particular be made, as examples of primary amines corresponding to the formula (V), of:
isopropylamine,
sec-butylamine,
tert-butylamine,
cyclohexylamine,
2,6-dimethylaniline,
2,6-diisopropylaniline,
2,6-dimethoxyaniline,
2,6-diisopropoxyaniline,
2,4,6-trimethylaniline,
2,4,6-triethylaniline,
1-aminonaphthalene,
2-aminonaphthalene.

The reaction of the compound of formula (I) and of the primary amine of formula (V) can be carried out according to basic catalysis, that is to say without catalyst, when the amine is sufficiently basic, that is to say when its pKa in water is greater than or equal to 10.

The reaction of the compound of formula (I) and of the primary amine of formula (V) can also be catalyzed by an acid. Thus, the reaction takes place in the presence of a catalytic amount of a strong protonic acid, that is to say, an acid exhibiting a pKa in water of less than 5, preferably of less than 1.

Mention may be made, as examples of strong protic acids, inter alia, of sulfuric acid, chlorosulfuric acid, perchloric acid, sulfonic acids such as, for example, methanesulfonic, trifluoromethanesulfonic, toluenesulfonic or phenolsulfonic acid, or carboxylic acids, such as acetic acid or trifluoroacetic acid.

Mention may be made, as other examples of protic acid catalysts, of sulfonic resins and more particularly resins sold under various trade names. Mention may be made, inter alia, of the following resins: Temex 50, Amberlyst 15, Amberlyst 35, Amberlyst 36, and Dowex 50W.

The abovementioned resins are composed of a polystyrene backbone which carries functional groups which are sulfonic groups. The polystyrene backbone is obtained by polymerization of styrene and divinylbenzene under the influence of an activating catalyst, generally an organic peroxide, which results in a crosslinked polystyrene which is subsequently treated with concentrated chlorosulfuric or sulfuric acid, resulting in a sulfonated styrene/divinylbenzene copolymer.

It is also possible to resort to sulfonic resins which are phenol/formol copolymers and which carry a methylenesulfonic group on the aromatic nucleus, for example, the resin sold under the name Duolite Arc 9359.

Other commercially available resins are also suitable and mention may be made of perfluorinated resins carrying sulfonic groups and more particularly carrying Nafion which is a copolymer of tetrafluoroethylene and perfluoro[2-(fluorosulfonylethoxy)propyl] vinyl ether.

Use is preferably made, among the abovementioned acids, of methanesulfonic acid or p-toluenesulfonic acid.

The amount of acid employed is preferably catalytic.

Thus, the amount of acid, expressed with respect to the amount of aldehyde, represents from 1 to 10% by weight, preferably from 1 to 5% by weight, of the weight of aldehyde, when the latter is in deficit with respect to the amine of formula (V).

The reaction is carried out in the presence of an organic or nonorganic solvent.

Mention may in particular be made, as examples of organic solvents suitable for the invention, of aliphatic, cycloaliphatic or aromatic hydrocarbons which may or may not be halogenated and more particularly hexane, heptane, isooctane, decane, benzene, toluene, methylene chloride or chloroform.

As the water formed during the reaction is removed, it is desirable to choose a solvent capable of forming an azeotrope with water. Toluene is thus preferably chosen.

The amount of solvent employed is such that the concentration of the compound of formula (I) varies between 5 and 50% by weight of the reaction mixture.

Thus, according to the invention, the strong acid is brought together in the presence of the mixture of the compound of formula (I) and of the amine of formula (V) in the organic solvent.

The reaction mixture is subjected to azeotropic distillation.

To this end, the distillation is carried out at a temperature of between 40° C. and 120° C., at atmospheric pressure or under a reduced pressure ranging from 10 mmHg up to atmospheric pressure, preferably between 10 and 200 mmHg.

The imine of formula (IV):

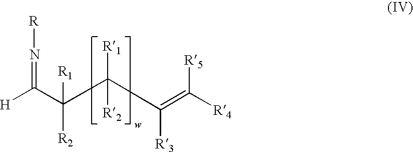

in said formula:
R, R₁, R₂, R′₁, R′₂, R′₃, R′₄, R′₅ and w have the meanings given above,
   is obtained in the organic solvent.

The imine is recovered by evaporation of the solvent, in particular by distillation under reduced pressure, for example, between 1 and 100 mmHg.

Another alternative embodiment of the invention consists in removing the water formed in the reaction using a dehydrating agent, for example magnesium sulfate or 4 Å molecular sieve (aluminosilicate).

Said agent is separated, preferably by filtration, and then the organic solvent is evaporated as mentioned above.

The invention is also targeted at the use of the compound of imine type of formula (IV) in the preparation of a cyclic iminium salt corresponding to the general formula (VI):

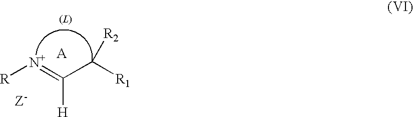

in said formula:
A represents a ring comprising 5 or 6 atoms, at least one of the atoms of which is a nitrogen atom as represented,
L is a divalent group corresponding to the following formula:

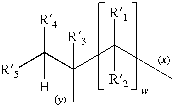

w is a number equal to 1 or 2,
R′₁, R′₂, R′₃, R′₄ and R′₅, which are identical or different, represent a hydrogen atom or an alkyl, cycloalkyl, aryl or aralkyl group,
(x) and (y) respectively pinpoint the two bonds established between the carbon atom carrying the R₁ and R₂ groups and the nitrogen atom carrying the R group,
R, R₁ and R₂ have the meanings given above,
Z represents an anion, such as, for example, a halide, preferably a chloride (in the Cl⁻ or HCl₂⁻ form or their mixture) or a bromide, or an acetate, trifluoroacetate, mesylate or tosylate group.

According to the process of the invention, the compound of formula (VI) is prepared from the linear iminium salt obtained by reaction of the compound of formula (IV) and a strong acid, followed by cyclization of the linear iminium salt obtained.

In accordance with the process of the invention, it is not necessary, in order to obtain the cyclic iminium salt, to isolate the imine of formula (IV) obtained as an intermediate or the linear iminium salt also obtained as an intermediate.

Recourse is had to a strong acid in order to form the linear iminium salt.

Mention may more particularly be made, as nonlimiting examples of strong protonic acids, of hydracids such as hydrochloric acid or hydrobromic acid, halogenated or non-halogenated oxyacids, such as sulfuric acid or perchloric acid, halogenated or non-halogenated sulfonic acids, such as fluorosulfonic acid, chloro-sulfonic acid, trifluoromethanesulfonic acid, methanesulfonic acid, ethanesulfonic acid, ethanedi-sulfonic acid, benzenesulfonic acid, benzenedisulfonic acids, toluenesulfonic acids, xylenesulfonic acids, naphthalenesulfonic acids and naphthalenedisulfonic acids, or halocarboxylic acids, such as in particular trichloroacetic acid.

Use is preferably made, among these acids, of hydrochloric acid.

Recourse is preferably had to concentrated acid solutions as the presence of water in the medium slows down the reaction kinetics.

For example, recourse is had to a 37% by weight hydrochloric acid solution or to a sulfuric acid solution of at least 95%, preferably of greater than 98%.

Use is advantageously made of hydrochloric acid in the gaseous form.

The amount of acid that is expressed by the ratio of the number of equivalents of protons to the number of moles of compound of formula (IV) can vary between approximately 1 and approximately 10 and preferably between 2 and 3.

The process of the invention is generally carried out at atmospheric pressure but preferably under a controlled atmosphere of inert gases. It is possible to establish an atmosphere of rare gases, preferably argon, but it is more economical to resort to nitrogen.

From a practical viewpoint, the compound of formula (IV) and an organic solvent are charged and then the acid is introduced, preferably a bubbling of hydrochloric acid.

The temperature of the reaction is advantageously between approximately −10° C. and approximately 20° C. and more preferably between −5° C. and 0° C.

The acid is added gradually, continuously or in fractions, over a period of time preferably varying between 3 and 8 hours.

At the end of the reaction, the linear iminium salt is obtained and is subsequently cyclized.

To this end, the reaction medium is brought to a reaction temperature preferably chosen between 60° C. and 100° C., and more preferably between 70° C. and 90° C.

After maintaining the chosen temperature for a period of time of 5 to 15 hours, the iminium salt of formula (VI) is obtained, which salt generally precipitates from the reaction medium.

It is separated according to conventional techniques for solid/liquid separation, preferably by filtration.

At the end of the reaction, the cyclized product which preferably corresponds to the following formula (VI):

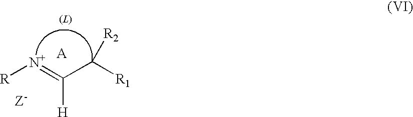

in said formula:
R, R₁, R₂, Z, A and L have the meanings given above,
   is obtained.

In the formula (VI), Z is preferably a chloride (in the Cl⁻ or HCl₂⁻ form or their mixture).

According to an additional stage of the process of the invention, the carbene is generated from the cyclic iminium salt of formula (VI) by reacting the latter with a strong base.

Recourse may be had in particular, as strong bases, to butyllithium, sodium tert-butoxide, potassium tert-butoxide, sodium amide or sodium hydride.

The strong base can be in the liquid or solid form, preferably in the solid form.

The amount of base, expressed with respect to the cyclic iminium salt of formula (VI), generally varies from the stoichiometric amount indeed up to an excess which can reach 200%.

The reaction is carried out at a low temperature of between −78° C. and 25° C.

The reaction is generally carried out at atmospheric pressure but preferably under a controlled atmosphere of inert gases. It is possible to establish an atmosphere of rare gases, preferably argon, but it is more economical to resort to nitrogen.

The reaction is carried out under anhydrous conditions and in a chosen aprotic organic solvent which is polar or nonpolar and inert under the conditions of the reaction.

Thus, it is preferable for the organic solvent to comprise less than 5 ppm of water.

Mention may be made, as examples of organic solvents, inter alia, of aliphatic, cycloaliphatic or aromatic hydrocarbons, more particularly hexane, heptane, isooctane, decane, benzene or toluene, or solvents of ether type, in particular diethyl ether, tetrahydrofuran or methyltetrahydrofuran.

The amount of organic solvent employed is such that the concentration of the cyclic iminium salt of formula (VI) varies between 10 and 50% of the weight of the reaction medium.

From a practical viewpoint, the base is introduced into the organic solvent comprising the cyclic iminium salt of formula (VI).

A carbene is obtained that can be symbolized by the formula (VII).

It should be noted that, depending on the use envisaged for the carbenes and more particularly in the case of the preparation of organometallic complexes, it is possible to generate the carbene in situ by combining the cyclic iminium salt of formula (VI) and a strong base as described above.

The carbene is recovered conventionally from the reaction medium.

In the case where the solvent is nonpolar (for example heptane, cyclohexane or toluene), the salts formed precipitate and are separated, for example, by filtration.

If the solvent is more polar, such as THF, it is possible to precipitate the salts formed by addition of a nonpolar solvent, such as, for example, toluene.

The carbene is found in the organic solvent and it is recovered after the evaporation of the solvent.

The process of the invention thus makes it possible to prepare carbenes of CAAC type which can be represented by the following formula:

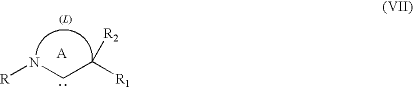

in said formula:
R, R₁ and R₂ have the meanings given above,
A represents a ring comprising 5 or 6 atoms, at least one of the atoms of which is a nitrogen atom as represented,
L is a divalent group corresponding to the following formula:

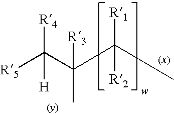

R′₁, R′₂, R′₃, R′₄, R′₅ and w have the meanings given above,
(x) and (y) respectively pinpoint the two bonds established between the carbon atom carrying the R₁ and R₂ groups and the nitrogen atom carrying the R group.

The CAAC carbenes preferably prepared according to the process of the invention correspond to the formula (VII) in which A represents a ring comprising 5 or 6 atoms and L represents a divalent group comprising 2 or 3 atoms.

Mention may be made, as more specific examples corresponding to this definition, of the carbenes corresponding to the following formulae (VIIa) and (VIIb):

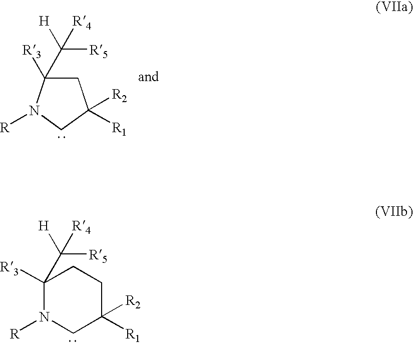

in said formulae:
R, R₁ and R₂ have the meanings given for the formula (I),
R′₃, R′₄ and R′₅, which are identical or different, represent a hydrogen atom or an alkyl, cycloalkyl, aryl or aralkyl group.

The carbenes which are even more preferred correspond to the formulae (VIIa) or (VIIb) in which R is an alkyl group or an optionally substituted aryl group, preferably an optionally substituted phenyl group; R₁ and R₂, which are identical or different, represent an alkyl group or an optionally substituted aryl group, preferably an optionally substituted phenyl group, or else R₁ and R₂ are bonded together to form a cycloalkane; and R′₃, R′₄ and R′₅, which are identical or different, represent an alkyl group, and R′₄ and R′₅ also represent a hydrogen atom.

The process of the invention is entirely well suited to preparing the CAAC carbenes which correspond to the formula (VIIa) or (VIIb) in which:
R represents a tert-butyl group, a phenyl group or a phenyl group substituted by 1 to 3 alkyl groups having from 1 to 4 carbon atoms,
R₁ and R₂, which are identical or different, represent a linear or branched alkyl group having from 1 to 4 carbon atoms or a phenyl group,
or R₁ and R₂ are bonded together to form a cyclopentane, a cyclohexane or a norbornane,
R′₃, R′₄ and R′₅, which are identical or different, represent a linear or branched alkyl group having from 1 to 4 carbon atoms and R′₄ and R′₅ also represent a hydrogen atom.

Examples of preferred carbenes are given below:

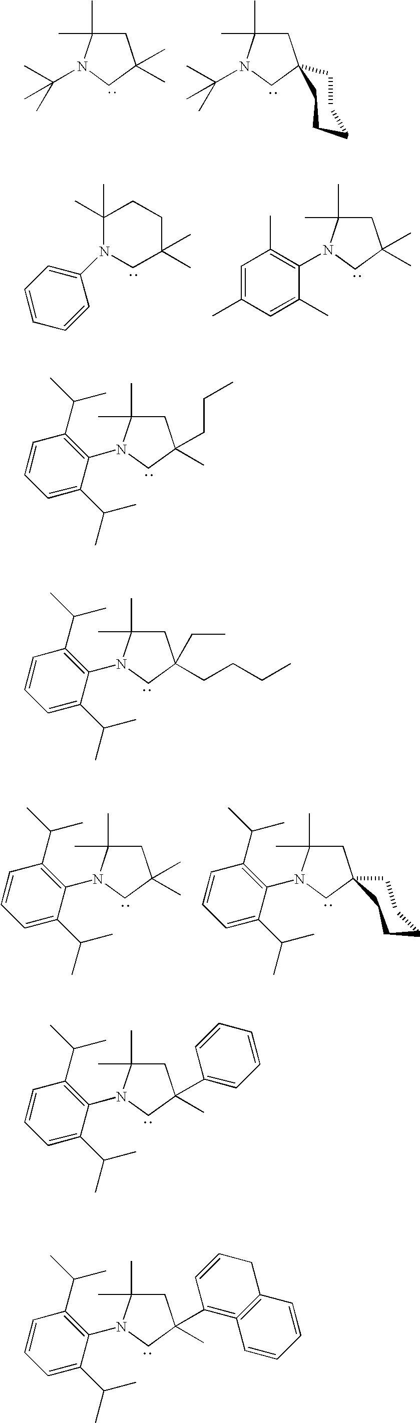

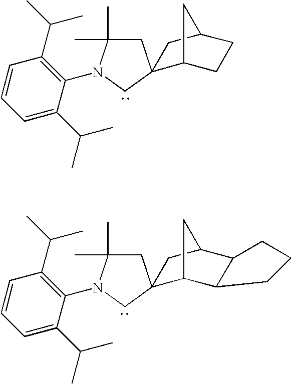

Another subject matter of the invention is, as novel products, the intermediates, iminium salt precursor imines, corresponding to the following formula:

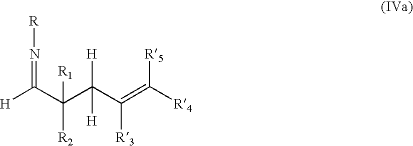

in said formula:
R represents a branched alkyl group, an aryl group or a substituted aryl group,
R₁ and R₂, which are identical or different, represent an alkyl group or an optionally substituted aryl group,
or R₁ and R₂ can be bonded together to form a spiro ring comprising from 3 to 18 atoms,
R′₃ represents a halogen atom, an alkyl group, an aryl group or a substituted aryl group,
R′₄ and R′₅, which are identical or different, represent a hydrogen atom, an alkyl group, an aryl group or a substituted aryl group.

In the formula (IVa), the aryl group is a phenyl or a naphthyl group and the substituted aryl group is a phenyl or naphthyl group substituted by one or more alkyl, alkoxy, alkenyl, alkynyl, cycloalkyl, aryl, aralkyl or amino groups, it being possible for the amino to be substituted by alkyl or cycloalkyl groups; a nitrile group; a halogen atom, preferably a chlorine or fluorine atom; or a haloalkyl group, preferably a perfluoromethyl group.

The preferred imines correspond to the formula (IVa) in which:
R represents a tert-butyl group; a phenyl group substituted by 3 methyl or ethyl groups in the o, o′ and p positions; a phenyl group substituted by 2 isopropyl or tert-butyl groups in the o and o′ positions; a phenyl group substituted by 3 isopropyl or tert-butyl groups in the o, o′ and p positions,
R₁ and R₂, which are identical or different, represent a methyl, ethyl, propyl, isopropyl, phenyl or substituted phenyl group,
or R₁ and R₂ can be bonded together to form a spiro ring comprising from 3 to 10 atoms, R′₃ represents a methyl group,
R′₄, and R′₅, which are identical or different, represent a hydrogen atom or a methyl group.

The preferred imines according to the invention correspond to the formula (IVa) in which at least one of the R₁ and R₂ groups represents an optionally substituted aryl group, preferably an optionally substituted phenyl group.

More preferably still, the R group represents an optionally substituted aryl group, preferably an optionally substituted phenyl group.

The invention is targeted more particularly at the imines corresponding to the formula (IVa) in which:
R represents a phenyl group substituted by 2 isopropyl groups in the o and o′ positions; or a phenyl group substituted by 3 methyl or isopropyl groups in the o, o′ and p positions,
R₁ and R₂, which are identical or different, represent a methyl group, a phenyl group or a substituted phenyl group,
or R₁ and R₂ can be bonded together to form a spiro ring comprising from 3 to 10 atoms,
R′₃ represents a methyl group,
R′₄ and R′₅ represent a hydrogen atom.

The preferred imines which comprise two aryl groups are represented diagrammatically by the formula (IVa₁):

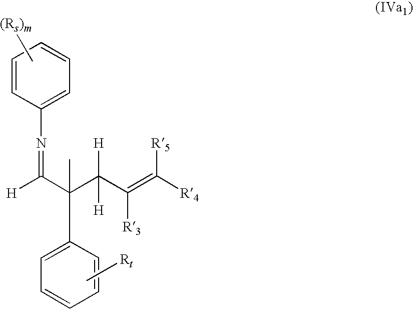

in said formula:
the Rₛ groups, which are identical or different, represent a substituent chosen from alkyl or alkoxy groups having from 1 to 4 carbon atoms,
m is the substituent number equal to 1, 2 or 3, preferably equal to 2 or 3,
Rₜ represents one or more substituents having the meaning given above,
R′₃ represents a methyl group,
R′₄ and R′₅ represent a hydrogen atom.

The invention is also targeted, as novel products, at iminium salts, which are precursors of carbenes, corresponding to the following formula:

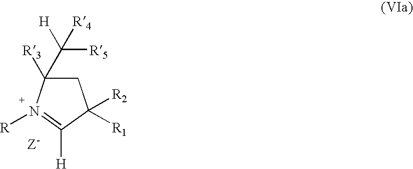

in said formula:
Z represents an anion, preferably a halide, chloride (in the Cl⁻ or HCl₂⁻ form or their mixture) or bromide; an acetate group; a trifluoroacetate group; a mesylate group; a tosylate group,
R represents a branched alkyl group, an aryl group or a substituted aryl group,
R₁ and R₂, which are identical or different, represent an alkyl group, an aryl group or a substituted aryl group,
or R₁ and R₂ can be bonded together to form a spiro ring comprising from 3 to 18 atoms,
R′₃ represents a hydrogen atom, an alkyl group, an aryl group or a substituted aryl group,
R′₄ and R′₅, which are identical or different, represent a hydrogen atom, an alkyl group, an aryl group or a substituted aryl group,
   except that R₁ and R₂ cannot be simultaneously two methyl groups or form a cyclohexane when R represents a 1,3-diisopropylphenyl group, and R₁ and R₂ cannot be a methyl and phenyl group or form a cyclohexane when R represents a tert-butyl group.

The preferred iminium salts correspond to the formula (VIa) in which:
R represents a tert-butyl group, a phenyl group substituted by 3 methyl or ethyl groups in the o, o′ and p positions; a phenyl group substituted by 2 isopropyl or tert-butyl groups in the o and o′ positions; a phenyl group substituted by 3 isopropyl or tert-butyl groups in the o, o′ and p positions,
R₁ and R₂, which are identical or different, represent a methyl, ethyl, propyl, isopropyl, phenyl or substituted phenyl group,
or R₁ and R₂ can be bonded together to form a spiro ring comprising from 3 to 10 atoms,
R′₃ represents a hydrogen atom or a methyl group,
R′₄ and R′₅, which are identical or different, represent a hydrogen atom or a methyl group.

More preferably still, the invention is targeted at the iminium salts corresponding to the formula (VIa) in which:
R represents a phenyl group substituted by 2 isopropyl groups in the o and o′ positions; or, a phenyl group substituted by 3 methyl or isopropyl groups in the o, o′ and p positions,
R₁ and R₂, which are identical or different, represent a methyl group, a phenyl group or a substituted phenyl group,
or R₁ and R₂ can be bonded together to form a spiro ring comprising from 3 to 10 atoms,
R′₃ represents a methyl group,
R′₄ and R′₅ represent a hydrogen atom.

The preferred iminium salts which comprise two aryl groups are represented diagrammatically by the formula (VIa₁):

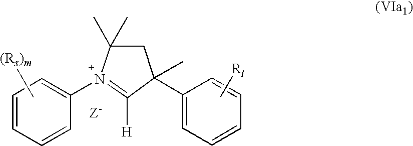

in said formula:
the Rₛ groups, which are identical or different, represent a substituent chosen from alkyl or alkoxy groups having from 1 to 4 carbon atoms,
m is the substituent number equal to 1, 2 or 3, preferably equal to 2 or 3,
Rₜ represents one or more substituents having the meaning given above,
R′₄ and R′₅ represent a hydrogen atom.

In the formulae (IVa₁) and (VIa₁), when m is equal to 2, the substituents are preferably in the ortho and ortho′ positions with respect to the carbon atom connected to the nitrogen atom and, when m is equal to 3, the substituents are in the ortho, ortho′ and para positions with respect to the carbon atom connected to the nitrogen atom.

In the formulae (VIa) and (VIa₁), Z is advantageously a chloride (in the Cl⁻ or HCl₂⁻ form or their mixture).

Implementational examples of the invention are given below by way of indication and without a limiting nature.

The yield given in the examples corresponds to the ratio of the number of moles of product formed to the number of moles of substrate involved.

### EXAMPLES

### Examples 1 to 8

In this series of tests, an α-disubstituted aldehyde corresponding to the formula (II) is allylated.

The procedure which is reproduced in the various examples is defined.

A solution of an unsaturated reactant which is an allyl halide (1.3 equivalent) and an α-disubstituted aldehyde (1 equivalent) is charged to a reactor equipped with a mechanical stirrer and a heating device (oil bath) and a mixture of a 50% by weight aqueous sodium hydroxide solution, an organic solvent and tetrabutylammonium bromide (4 mol %) is added dropwise to this solution at 70-80° C. (oil bath).

The organic solvent is toluene in all the examples except for examples 2 and 3 where it is 2-isopropylbenzene.

The mixture is stirred at 70-80° C. for 4.5 h, cooled to ambient temperature and then extracted with distilled water.

The aqueous phase is extracted with the solvent used and then the organic phase is dried on MgSO₄ and filtered through a sintered glass No. 4 filter.

The solvent is evaporated from the filtrate under a reduced pressure of approximately 50 mmHg (Rotavapor) then the fitrate is distilled under reduced pressure.

The results obtained are recorded in table (I).

TABLE (I)

| | | | | Isolated yield |
|---|---|---|---|---|
| | | | | Boiling point (° C.) |
| Ex. | Aldehyde | Allyl halide | Product obtained | Characteristics |
| 1 | 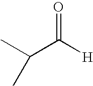 | 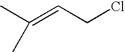 | 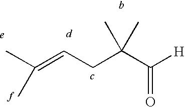 | Yd = 77% B.p. = 170° C. ¹H NMR δ ppm 9.38 (s, 1H, Ha) 4.96 (m, 1H, Hd) 2.05 (d, 2H, Hc) 1.59 (s, 1H, Hf) 1.50 (s, 1H, He) 0.94 (s, 6H, Hb) IR (cm⁻¹) 1728 (ν C═O) 882 (ν CH═C) |
| 2 | 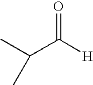 | 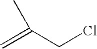 | 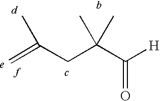 | Yd = 61% B.p. = 130° C. ¹H NMR δ ppm 9.40 (s, 1H, Ha) 4.7 (s, 1H, He) 4.5 (s, 1H, Hf) 2.1 (s, 2H, Hc) 1.5 (s, 3H, Hd) 0.92 (s, 6H, Hb) IR (cm⁻¹) 1720 (ν C═O) 910 (ν CH═CH2) |
| 3 | 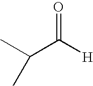 | 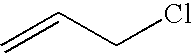 | 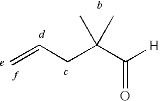 | Yd = 61% B.p. = 105° C. ¹H NMR δ ppm 9.30 (s, 1H, Ha) 4.92 (d, 1H, He) 4.88 (d, 1H, Hf) 2.05 (d, 1H, Hc) 1.08 (d, 1H, Hd) 1.08 (d, 1H, Hd) 0.88 (s, 6H, Hb) |
| 4 | 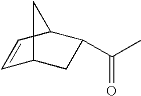 | 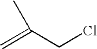 | 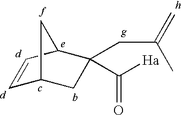 | Yd = 79% B.p. = 48° C./0.8 mbar Endo + exo ¹H NMR δ ppm 9.7 (s, 1H, Ha end) 9.4 (s, 1H, Ha exo) 6.1 (2s, 2H, Hd) 4.88 (s, 1H, Hh) 4.81 (s, 1H, Hh) 2.49 (s, 1H, He) 2.32 (s, 1H, Hc) 2.24 (d d, 2H, Hg) 1.65 (s, 2H, Hf) 1.55 (s, 3H, Hi) |
| 5 | 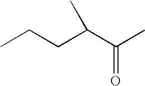 | 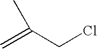 | 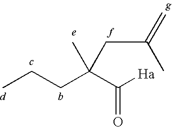 | Yd = 72% B.p. = 23− 25° C./0.8 mbar ¹H NMR δ ppm 9.44 (s, 1H, Ha) 4.75 (s, 1H, Hh) 4.58 (s, 1H, Hg) 2.23 (dd, 2H, Hf) 1.67 (s, 3H, Hi) 1.54 (m, 2H, Hc) 1.37 (m, 2H, Hb) 0.94 (s, 3H, He) 0.81 (t, 3H, Hd) |
| 6 | 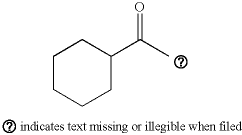 | 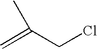 | 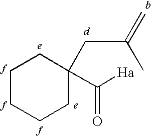 | Yd = 89% B.p. = 36− 38° C./0.8 mbar ¹H NMR δ ppm 9.4 (s, 1H, Ha) 4.7 (s, 1H, Hb) 4.5 (s, 1H, Hb) 2.1 (s, 2H, Hd) 1.7 (s, 3H, Hc) 1.53 (s, 4H, He) 1.2 (m, 6H, Hf) |
| 7 | 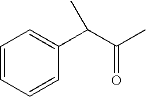 | 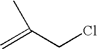 | 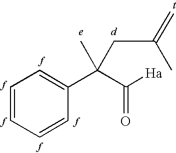 | Yd = 80% B.p. = 62− 64° C./0.8 mbar ¹H NMR δ ppm 9.49 (s, 1H, Ha) 7.25 (m, 5H, Hf) 4.76 (s, 1H, Hb) 4.58 (s, 1H, Hb) 2.65 (dd, 2H, Hd) 1.42 (s, 3H, Hc) 1.36 (s, 3H, He) |
| 8 | 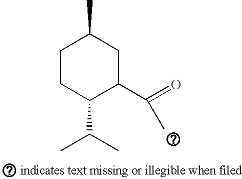 | 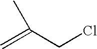 | 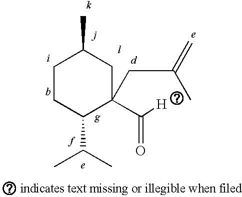 | Yd = 85% B.p. = 170° C. ¹H NMR δ ppm 9.52 (s, 1H, Ha) 4.91 (s, 1H, Hc) 4.63 (s, 1H, Hc) 2.34 (dd, 2H, Hd) 1.82 (m, 1H, Hf) 1.71 (s, 3H, Hb) 1.68 (m, 2H, Hl) 1.63 (m, 1H, Hg) 1.61 (m, 1H, Hj) 1.52 (m, 2H, Hi) 1.27 (m, 2H, Hh) 1.06 (d, 3H, Hk) 0.90 (d, 6H, He) |

### Examples 9 to 15

In this series of tests, an imine corresponding to the formula (IV) is synthesized by reacting an aromatic amine with a compound corresponding to the formula (I) which is an unsaturated aldehyde.

The procedure which is used in all the examples is given below.

The mixture of unsaturated aldehyde (1.1 equivalents) and the amine (1 equivalent) is brought together with catalytic p-toluenesulfonic acid (2 mol %) in toluene, as solvent, in a single-necked flask surmounted by Dean and Stark apparatus and a reflux condenser.

The solution is brought to reflux of toluene.

The formation of water is observed in the Dean and Stark apparatus.

The toluene is subsequently evaporated under a reduced pressure of approximately 50 mmHg (Rotavapor) and then the residue is distilled under reduced pressure.

The results obtained are reported in table (II).

TABLE (II)

| | | | | Isolated yield |
|---|---|---|---|---|
| | Unsaturated | | | Boiling point (° C.) |
| Ex | aldehyde | Amine | Product | Characteristics |
| 9 | 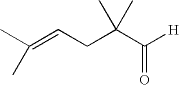 | 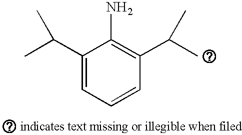 | 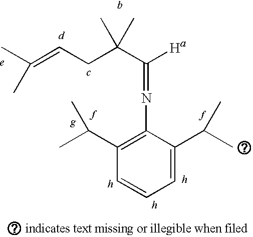 | Yd = 73% B.p. = 78-80° C./ 0.01 mbar ¹H NMR δ (mmp) 7.52(s, 1H, Ha) 7.1(m, 3H, Hb) 5.28(m, 1H, Hd) 2.95(m, 2H, Hf) 2.28(d, 2H, Hc) 1.75(s, 3H, He) 1.66(s, 3H, He) 1.24(s, 6H, Hb) 1.19(m, 12H, Hg) |
| 10 | 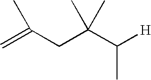 | 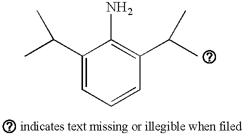 | 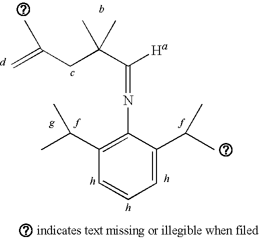 | Yd = 84.6% B.p. = 88-90° C./ 0.01 mbar ¹H NMR δ (ppm) 7.62(s, 1H, Ha) 7.16(m, 3H, Hh) 4.96(s, 1H, Hd) 4.82(s, 1H, Hd) 2.99(m, 2H, Hf) 2.37(s, 2H, Hc) 1.88(s, 3H, He) 1.32(s, 6H, Hb) 1.20(m, 12H, Hg) |
| 11 | 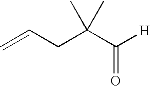 | 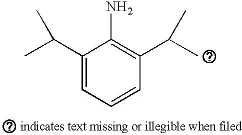 | 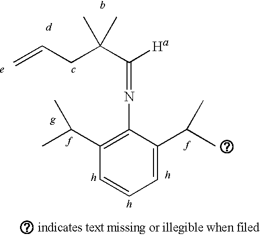 | Yd = 75% T_{ab} = 80° C./ 0.01 mbar ¹H NMR δ (ppm) 7.54(s, 1H, Ha) 7.10(m, 3H, Hh) 6.01(m, 1H, Hd) 5.15(s, 1H, He) 5.09(s, 1H, He) 2.92(m, 2H, Hf) 2.35(d, 2H, Hc) 1.24(s, 6H, Hb) 1.17(m, 12H, Hg) |
| 12 | 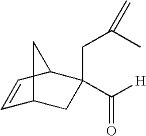 | 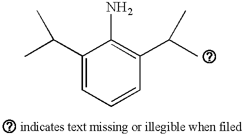 | 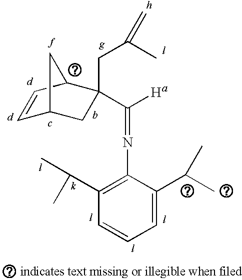 | Yd = 70% Flashed product ¹H NMR δ (ppm) 7.67(s, 1H, Ha)end 7.51(s, 1H, Ha)exo 7.09(m, 3H, Hl) 6.19(s, 2H, Hd) 4.80(s, 1H, Hh) 4.70(s, 1H, Hh) 2.49(s, 1H, He) 2.32(s, 1H, Hc) 2.21(m, 1H, Hg) 1.65(s, 2H, Hf) 1.10(s, 12H, Hj) |
| 13 | 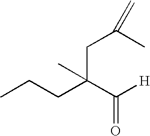 | 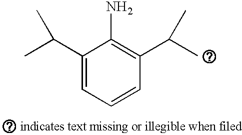 | 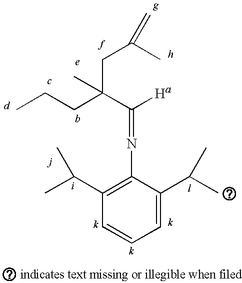 | Yd = 62.5% Flashed product ¹H NMR δ (ppm) 7.61(s, 1H, Ha) 7.13(m, 3H, Hk) 4.94(s, 1H, Hg) 4.81(s, 1H, Hg) 2.99(m, 2H, Hi) 2.39(s, 2H, Hf) 1.86(s, 3H, Hh) 1.61(m, 2H, Hc) 1.48(m, 2H, Hb) 1.30(s, 3H, He) 1.19(m, 12H, Hj) 0.99(t, 3H, Hd) |
| 14 | 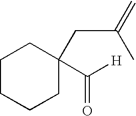 | 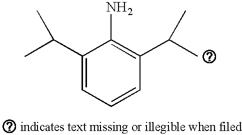 | 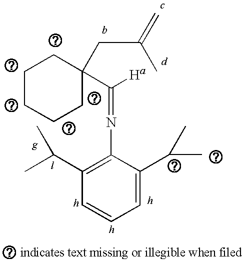 | Yd = 62% ¹H NMR δ (ppm) 7.59(s, 1H, Ha) 7.13(m, 3H, Hh) 4.94(s, 1H, Hc) 4.79(s, 1H, Hc) 3.05(m, 2H, Hf) 2.35(s, 2H, Hb) 1.85(s, 3H, Hd) 1.65(m, 10H, He) 1.10(d, 12H, Hg) IR (cm⁻¹) 1653.9 (ν C═N) 894.1 (ν C═C) |
| 15 | 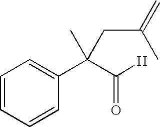 | 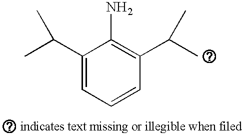 | 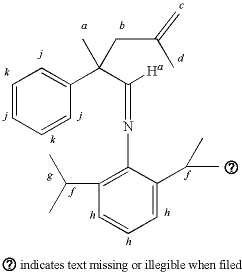 | Yd = 65% Flashed product ¹H NMR δ (ppm) 7.66(s, 1H, Ha) 7.40(m, 2H, Hk) 7.30(m, 2H, Hj) 7.20(m, 1H, Hl) 4.79(s, 1H, Hc) 4.63(s, 1H, Hc) 2.93(m, 2H, Hf) 2.88(m, 2H, Hb) 1.65(s, 3H, Hd) 1.33(s, 3H, He) 1.10(s, 12H, Hg) IR (cm⁻¹) 1654 (ν C═N) 892 (ν C═C) |

### Example 10

In this example, an imine corresponding to the formula (IV) is synthesized by reacting an aliphatic amine with a compound corresponding to the formula (I).

The unsaturated aldehyde (1 molar equivalent) and tert-butylamine (1.1 molar equivalents) mixture is dissolved in the solvent, toluene, in the presence of 4 Å molecular sieve in a single-necked flask surmounted by a reflux condenser.

The medium is subsequently filtered and the filtrate is evaporated under a reduced pressure of approximately 50 mmHg (Rotavapor) and then distilled under reduced pressure.

The results obtained are recorded in table (III).

TABLE (III)

| | | | | Isolated yield |
|---|---|---|---|---|
| | Unsaturated | | | Boiling point (° C.) |
| Ex | aldehyde | Amine | Product | Characteristics |
| 16 | 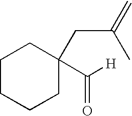 | 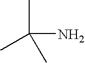 | 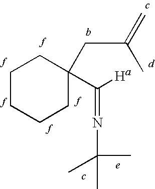 | Yd = 67% T_{bp} = 48-50° C./ 0.01 mbar ¹H NMR δ (ppm) 7.32(s, 1H, Ha) 4.67(s, 1H, Hc) 4.54(s, 1H, Hc) 2.04(s, 2H, Hb) 1.56(s, 3H, Hd) 1.30(m, 10H, Hf) 1.07(s, 9H, He) |

### Example 17

In this example, the synthesis is carried out of the cyclic iminium corresponding to the general formula (VI):

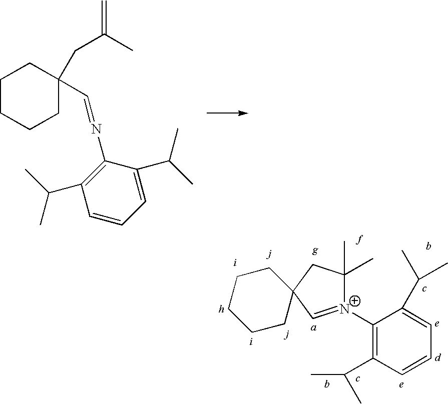

The imine is dissolved in toluene (solvent) and the solution is cooled to 0° C.

Bubbling of HCl gas into the solution at 0° C. is maintained for 5 hours.

A change in color of the reaction medium from pale yellow to dark yellow is noted.

The reaction medium is then heated at 80° C. for 12 hours; a white precipitate is observed (glass wool).

Filtration through a sintered glass No. 4 filter and washing with ether makes it possible to recover a fine white powder which corresponds to the cyclized product.

The yield obtained is 80%.

The characteristics of the product obtained are as follows:

melting point=242-243° C.

¹H NMR δ (ppm)

10.55 (s, 1H, Ha)

7.41 (m, 1H, Hd)

7.25 (m, 2H, He)

2.62 (m, 2H, He)

2.50 (m, 2H, Hh)

2.37 (s, 2H, Hg)

1.87 (d, 4H, Hi)

10.65 (d, 4H, Hi)

1.62 (s, 1H, Hf)

1.27 (d, 6H, Hb)

1.19 (d, 6H, Hb)

### Example 18

In this example, the synthesis is carried out of the cyclic iminium corresponding to the general formula (VI):

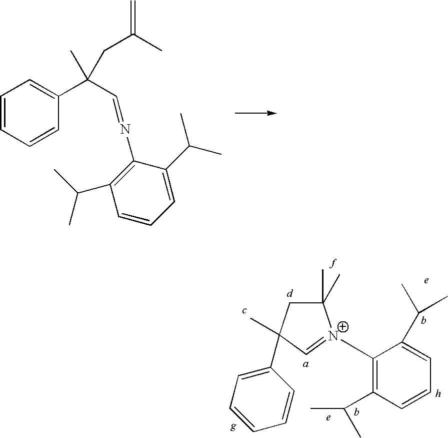

The imine is dissolved in toluene and the solution is cooled to 0° C.

Bubbling the HCl gas into the solution at 0° C. is maintained for 5 hours.

The reaction medium is then heated at 80° C. for 12 hours; no precipitate is observed but a change in color of the reaction medium is observed.

The evaporation of the toluene to dryness under a reduced pressure of approximately 50 mmHg (Rotavapor) results in a white powder which is taken up in ether and filtered through a sintered glass No. 4 filter.

This white powder corresponds to the cyclized product.

The yield obtained is 79%.

The characteristics of the product obtained are as follows:

melting point=204-205° C.

¹H NMR δ (ppm)

11.9 (s, 1H, Ha)

7.36 (m, 5H, Hg)

7.21 (m, 3H, Hh)

3.14 (d, 2H, Hd)

2.62 (m, 2H, Hb)

1.92 (s, 3H, Hc)

1.45 (s, 6H, Hf)

1.27 (d, 12H, He)

### Examples 19 to 23

The following examples relate to other cyclizations in an acidic medium which are carried out while reproducing the following procedure.

The imine is dissolved in toluene and the solution is cooled to 0° C.

2 molar equivalents of HCl in ether are introduced.

The mixture is then gradually brought to 80° C.

The evaporation of the toluene to dryness under a reduced pressure of approximately 50 mmHg (Rotavapor) results in a white powder which is taken up in ether and filtered through a sintered glass No. 4 filter.

This white powder corresponds to the cyclized product.

The results obtained are recorded in table (IV).

TABLE (IV)

| | | | | Mol | Volume | | |
|---|---|---|---|---|---|---|---|
| | | Weight | | HCl/Vol | of | | Iso- |
| | | (g) | | 2M | toluene | Reaction | lated |
| Ex | Starting imine | Mol | Cyclized product | HCl/Et₂O | (ml) | time (h) | Yd (%) |
| 19 | 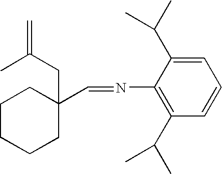 | 9 g 0.028 mol | 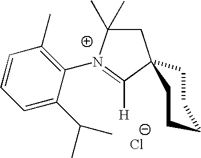 | 0.056 mol 28 ml | 170 | 21 | 89 |
| 20 | 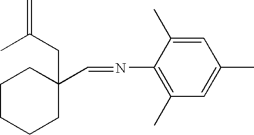 | 6.5 g 0.023 mol | 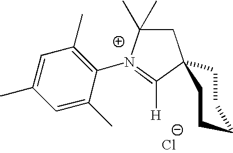 | 0.046 mol 23 ml | 170 | 16 | 77 |
| 21 | 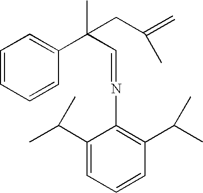 | 15 g 0.043 mol | 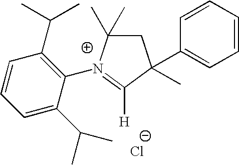 | 0.086 mol 43 ml | 100 | 15 | 98 |
| 22 | 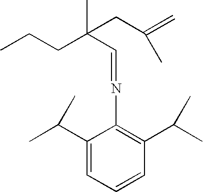 | 15 g 0.048 mol | 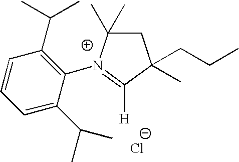 | 0.096 mol 48 ml | 100 | 15 | 84 |
| 23 | 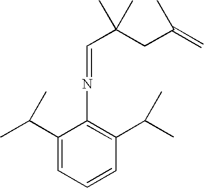 | 43 g 0.151 mol | 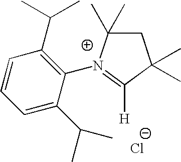 | 0.301 mol 150 ml | 150 | 18 | 90 |

### Example 24

1.05 g (2.90 mmol) of the cyclic iminium salt having the formula given below:

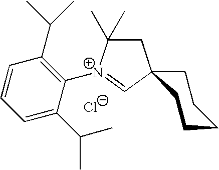

0.17 g (3.26 mmol) of a sodium amide and 0.02 g of t-BuOK are introduced into a dry 100 ml reactor under argon.

The temperature is lowered to −78° C. and 25 ml of dry THF are added.

The mixture is left to react at −78° C. for 1 hour and at 25° C. for 16 hours.

The THF is evaporated under a reduced pressure of 10 mmHg.

Dry toluene is added.

The mixture is filtered under argon pressure and the toluene solution is recovered, which solution contains exclusively the following carbene, the NMR analyses of which confirm the structure.

1.-24. (canceled)

25. A process for the preparation of a precursor of a CAAC carbene of formula (VI): 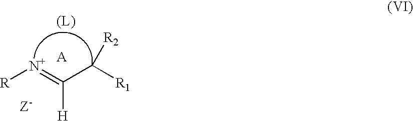
wherein:
R is an alkyl, cycloalkyl, aryl, aralkyl or heteroaryl radical;
R₁ and R₂, which may be identical or different, are each an alkyl, alkenyl, alkynyl, cycloalkyl, aryl, aralkyl, alkoxy, alkenyloxy, alkynyloxy, aryloxy or alkoxycarbonyl radical, with the proviso that R₁ and R₂ may together form a spiro ring member having from 3 to 18 atoms;
A is a ring member having 5 or 6 atoms, at least one of which is a nitrogen atom;
L is a divalent radical having the following formula: 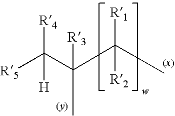
wherein:
w is a number equal to 1 or 2;
R′₁, R′₂, R′₃, R′₄ and R′₅, which may be identical or different, are each a hydrogen atom or an alkyl, cycloalkyl, aryl or aralkyl radical;
(x) and (y) respectively define the two bonds established between the carbon atom bearing the R₁ and R₂ groups and the nitrogen atom bearing the R group;
Z is an anion;
comprising reacting a compound of formula (I): 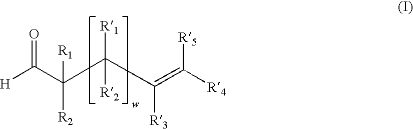
wherein:
R₁, R₂, R′₁, R′₂, R′₃, R′₄, R′₅ and w are as defined above with a primary amine of formula (V):
R—NH₂ (V)
wherein:
R is an alkyl, cycloalkyl, aryl, aralkyl or heteroaryl radical to form an imine having the formula (IV): 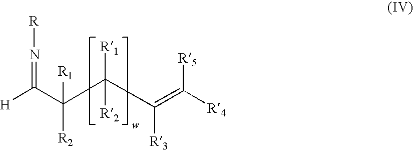
wherein:
R, R₁, R₂, R′₁, R′₂, R′₃, R′₄, R′₅ and w are as defined above.

26. The process as defined by claim 25, comprising preparing the intermediate compound of formula (I) by reacting an aldehyde having at least one hydrogen atom in the α position with respect to the carbonyl group with an unsaturated reactant, carrying a leaving group, in a two-phase medium, in the presence of a strong base and of a phase transfer catalyst.

27. The process as defined by claim 26, comprises preparation of the intermediate compound of formula (I) by reaction:
of an aldehyde of formula (II): 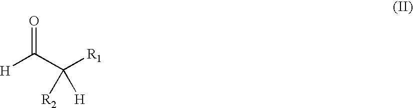
wherein:
R₁ and R₂, which may be identical or different, are each an alkyl, alkenyl, alkynyl, cycloalkyl, aryl, aralkyl, alkoxy, alkenyloxy, alkynyloxy, aryloxy or alkoxycarbonyl radical, with the proviso that R₁ and R₂ may together form a spiro ring member having from 3 to 18 atoms, with an unsaturated reactant bearing a leaving group (III): 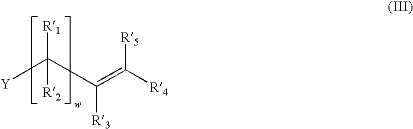
wherein:
w is a number equal to 1 or 2;
R′₁, R′₂, R′₃, R′₄ and R′₅, which may be identical or different, are each a hydrogen atom or an alkyl, cycloalkyl, aryl or aralkyl radical; and
Y is a leaving group selected from among bromine, chlorine or a sulfonic ester group of formula —OSO₂—Rₑ, in which Rₑ is a hydrocarbon radical.

28. The process as defined by claim 26, wherein the base is an inorganic base, comprising an alkali metal or alkaline earth metal hydroxide, or an alkali metal phosphate or hydrogen phosphate, or an organic base.

29. The process as defined by claim 26, wherein the phase transfer catalyst is an onium salt which comprises an onium ion having the following general formula: 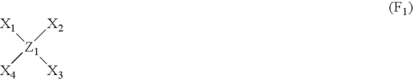
wherein:
Z₁ is N or P; and
X₁, X₂, X₃ and X₄, which may be identical or different, are each:
a linear or branched alkyl radical having from 1 to 16 carbon atoms and is optionally substituted by one or more phenyl, hydroxyl, halogen, nitro, alkoxy or alkoxycarbonyl groups or atoms, the alkoxy radicals having from 1 to 4 carbon atoms;
a linear or branched alkenyl radical having from 2 to 12 carbon atoms;
an aryl radical having from 6 to 10 carbon atoms and is optionally substituted by one or more alkyl radicals having from 1 to 4 carbon atoms, alkoxy radicals, alkoxycarbonyl radicals, the alkoxy radical having from 1 to 4 carbon atoms, or halogen atoms, with the proviso that two of said groups X₁ to X₄ may together form a linear or branched alkylene, alkenylene or alkadienylene radical having from 3 to 6 carbon atoms.

30. The process as defined by claim 29, wherein the phase transfer catalyst comprises tributylbenzylammonium or -phosphonium chloride or bromide, tetramethylammonium or -phosphonium chloride or bromide, tetraethylammonium or -phosphonium chloride or bromide, tetrabutylammonium or -phosphonium chloride or bromide, or TAED.

31. The process as defined by claim 25, comprising preparing a compound of imine type of formula (IV) by reacting a compound of formula (I) with a primary amine of formula (V) in the presence of a strong protonic acid, optionally methanesulfonic acid or p-toluenesulfonic acid.

32. The process as defined by claim 25, wherein the amine of formula (V) is selected from among isopropylamine, sec-butylamine, tert-butylamine, cyclohexylamine, 2,6-dimethylaniline, 2,6-diisopropylaniline, 2,6-dimethoxyaniline, 2,6-diisopropoxyaniline, 2,4,6-trimethylaniline, 2,4,6-triethylaniline, 1-aminonaphthalene or 2-aminonaphthalene.

33. The process as defined by claim 25, wherein the water formed during the reaction is removed by azeotropic distillation or using a dehydrating agent, optionally magnesium sulfate or 4 Å molecular sieve (aluminosilicate).

34. The process as defined by claim 25, comprising a stage of cyclization of the compound of formula (IV) to obtain a cyclic iminium salt of formula (VI):

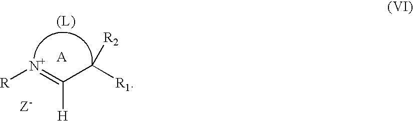

35. The process as defined by claim 34, wherein the compound of formula (VI), Z is a halide, or an acetate, trifluoroacetate, mesylate or tosylate group.

36. The process as defined by claim 34, wherein the compound of formula (VI) is obtained from the linear iminium salt prepared by reacting the compound of formula (IV) with a strong acid, optionally in the gaseous state, followed by cyclization of the linear iminium salt obtained.

37. A process for the preparation of a carbene of CAAC type having the following formula (VII): 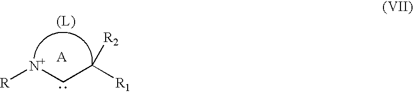
wherein:
R, R₁ and R₂ are as defined above in claim 25;
A is a ring member having 5 or 6 atoms, at least one of the atoms of which is a nitrogen atom;
L is a divalent group having the following formula: 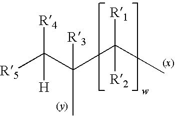
R′₁, R′₂, R′₃, R′₄, R′₅ and w are also as defined in claim 25;
(x) and (y) respectively define the two bonds established between the carbon atom bearing the R₁ and R₂ groups and the nitrogen atom bearing the R group;
comprising reacting the cyclic iminium salt of formula (VI) as defined by claim 25 with a strong base in an aprotic organic solvent under anhydrous conditions.

38. The process as defined by claim 37, wherein the carbene of CAAC type obtained has either of the following formulae (VIIa) and (VIIb): 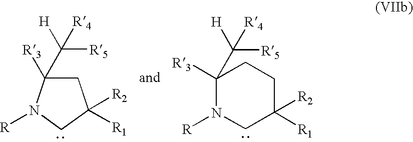
wherein:
R, R₁ and R₂ have the meanings given for formula (I); and
R′₃, R′₄ and R′₅, which may be identical or different, are each a hydrogen atom or an alkyl, cycloalkyl, aryl or aralkyl radical.

39. The process as defined by claim 38, wherein the carbene of CAAC type obtained corresponds to either of the formulae (VIIa) and (VIIb) in which:
R is a tert-butyl radical, a phenyl radical or a phenyl radical substituted by one to three alkyl radicals having from 1 to 4 carbon atoms,
R₁ and R₂, which may be identical or different, are each a linear or branched alkyl radical having from 1 to 4 carbon atoms or a phenyl radical;
or R₁ and R₂ may together form a cyclopentane, a cyclohexane or a norbornane; and
R′₃, R′₄ and R′₅, which may be identical or different, are each a linear or branched alkyl radical having from 1 to 4 carbon atoms and R′₄ and R′₅ are each a hydrogen atom.

40. An imine, which is an iminium salt precursor, having the following formula: 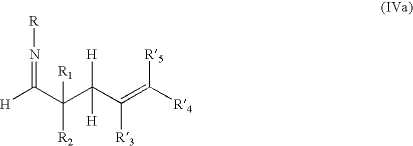
wherein:
R is a branched alkyl radical, an aryl radical or a substituted aryl radical;
R₁ and R₂, which may be identical or different, are each an alkyl radical or an optionally substituted aryl radical, with the proviso that R₁ and R₂ may together form a spiro ring member having from 3 to 18 atoms;
R′₃ is a hydrogen atom, an alkyl radical, an aryl radical or a substituted aryl radical; and
R′₄ and R′₅, which may be identical or different, are each a hydrogen atom, an alkyl radical, an aryl radical or a substituted aryl radical.

41. The imine which is an iminium salt precursor as defined by claim 40, having the formula (IVa) in which the aryl radical is a phenyl or a naphthyl radical and the substituted aryl radical is a phenyl or naphthyl radical substituted by one or more alkyl, alkoxy, alkenyl, alkynyl, cycloalkyl, aryl, aralkyl or amino groups, with the proviso that the amino group may be substituted by alkyl or cycloalkyl radicals, a nitrile group, a halogen atom, or a haloalkyl radical.

42. The imine which is an iminium salt precursor as defined by claim 40, having the formula (IVa) in which:
R is a tert-butyl radical, a phenyl radical substituted by 3 methyl or ethyl groups in the o, o′ and p positions, a phenyl radical substituted by 2 isopropyl or tert-butyl groups in the o and o′ positions, or a phenyl radical substituted by 3 isopropyl or tert-butyl groups in the o, o′ and p positions;
R₁ and R₂, which may be identical or different, are each a methyl, ethyl, propyl, isopropyl, phenyl or substituted phenyl radical, with the proviso that R₁ and
R₂ may together form a Spiro ring member having from 3 to 10 atoms,
R′₃ is a methyl radical; and
R′₄ and R′₅, which may be identical or different, are each a hydrogen atom or a methyl radical.

43. The imine which is an iminium salt precursor as defined by claim 40, having the formula (IVa) in which:
R is a phenyl radical substituted by 2 isopropyl groups in the o and o′ positions; or a phenyl radical substituted by 3 methyl or isopropyl groups in the o, o′ and p positions;
R₁ and R₂, which may be identical or different, are each a methyl radical, a phenyl radical or a substituted phenyl radical, with the proviso that R₁ and R₂ may together form a spiro ring member having from 3 to 10 atoms;
R′₃ is a methyl radical; and
R′₄ and R′₅ are each a hydrogen atom.

44. An iminium salt which is a carbene precursor, having the following formula: 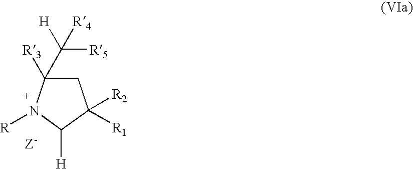
wherein:
Z is an anion, optionally a halide, chloride (in the Cl⁻ or HCl₂″ form or their mixture) or bromide, an acetate group, a trifluoroacetate group, a mesylate group, or a tosylate group;
R is a branched alkyl radical, an aryl radical or a substituted aryl radical;
R₁ and R₂, which may be identical or different, are each an alkyl radical, an aryl radical or a substituted aryl radical, with the proviso that R₁ and R₂ may together form a spiro ring member having from 3 to 18 atoms;
R′₃ is a hydrogen atom, an alkyl radical, an aryl radical or a substituted aryl radical;
R′₄ and R′₅, which may be identical or different, are each a hydrogen atom, an alkyl radical, an aryl radical or a substituted aryl radical;
except that R₁ and R₂ cannot simultaneously be two methyl radicals or form a cyclohexane ring when R is a 1,3-diisopropylphenyl radical, and R₁ and R₂ cannot be a methyl and phenyl radical or form a cyclohexane ring when R is a tert-butyl radical.
